(19) United States
(12) Patent Application Publication
(10) Pub. No.: US 2023/0287523 A1
(43) Pub. Date: Sep. 14, 2023

(54) SARS-COV-2 RAPID SEQUENCE-BASED DIAGNOSTIC ASSAY WITH SENSORS OF IMMUNE ACTIVATION AND THE VIRAL MICROBIOTA

(71) Applicant: Ohio State Innovation Foundation, Columbus, OH (US)

(72) Inventors: Daniel JONES, Columbus, OH (US); Huolin TU, Lewis Center, OH (US); Matthew AVENARIUS, Columbus, OH (US)

(21) Appl. No.: 18/040,523

(22) PCT Filed: Aug. 6, 2021
(86) PCT No.: PCT/US2021/044915

§ 371 Date: Feb. 3, 2023

Related U.S. Application Data
(60) Provisional application No. 63/061,999, filed on Aug. 6, 2020.

(51) Int. Cl. C12Q 1/70

(52) U.S. Cl. CPC C12Q 1/701; C12Q 2600/158; C12Q 2600/16

(57) ABSTRACT

Disclosed are assays, kits, primers, and methods for detecting SARS-CoV-2. The assays, kits, primers, and methods can also include sensors of immune activation and viral microbiota.

### I. CROSS-REFERENCE TO RELATED APPLICATIONS

This application claims benefit of U.S. Provisional Application No. 63/061,999, filed Aug. 6, 2020, incorporated herein by reference in its entirety.

### II. BACKGROUND

Diagnosis of SARS-CoV-2 infection, which is responsible for COVID-19 disease, is typically performed by molecular analysis of upper respiratory sampling (e.g. nasal swab). Transcriptional-mediated amplification (TMA) and quantitative (real-time) polymerase chain reaction (qPCR) using one or more viral targets are the predominant diagnostic methodologies currently used to detect these infections. Each of these methodologies have differing sensitivities of virus detection leading to discordances, equivocal results and possible false-positive and false-negative results. Many of these discordances are believed to result from undetected problems with sample adequacy (e.g. poor quality swabs).

The first step in managing COVID-19 is the rapid and accurate detection of SARS-CoV-2 enabled by real-time reverse transcription-polymerase chain reaction (RT-PCR)7. RT-PCR detects SARS-CoV-2 nucleic acids present in nasopharyngeal fluids. Testing is used to prevent infectious spread between persons and communities that include asymptomatic infected persons, whose viral shedding can inadvertently spread the infection to the elderly and those with disease comorbidities9. Accurate viral detection is a starting point to contain the COVID-19 pandemic. Lapses affect public safety, enabling infection spread aided by false-negative test results. Improving test sensitivity and specificity remains an urgent need.

In throat swabs and sputum, the viral shedding peaks at five to six days after symptom onset and ranges from 10⁴ to 10⁷ copies ml⁻¹. This reflects higher virus levels in the respiratory tract. The viral RNA detection rate in nasal swabs of infected people has approached 100%. The positivity rates for blood, saliva and tears are 88, 78 and 16%, respectively. The self-collection of naso- or oropharyngeal swabs facilitates large-scale population field testing employing the chemiluminescence immunoassay and the enzyme-linked immunosorbent and lateral-flow immunochromatographic assays. The lateral-flow immunochromatographic assay uses gold nanoparticles (AuNPs) and a colorimetric label to provide a rapid platform for point-of-contact serological detection. Here, SARS-CoV-2-specific antigen is conjugated with nanoparticles. By blood or saliva specimen loading, SARS-CoV-2 IgG and IgM can bind to the SARS-CoV-2 antigen and antibody, which is detected colorimetrically. The assay is completed in 20 minutes with a ˜90% accuracy. To date, the minimum length of viral shedding is 7 days after symptom onset, with viral infectivity observed within 24 hours. SARS-CoV-2 detection declines to undetectable levels, paralleling the presence of serum neutralizing antibodies. Even among cases with concurrent high viral loads, the live virus could not be propagated in cell culture 8 days after symptom onset (Kevadiya, B. D., Machhi, J., Herskovitz, J. et al. Diagnostics for SARS-CoV-2 infections. Nat. Mater. 20, 593-605 (2021)).

What are needed are new tools and methods for the complete characterization of the type and level of SARS-CoV-2 infection which provide a more clinically useful test result to understand the stage and severity of disease and the appropriate therapeutic intervention.

### III. SUMMARY

Disclosed herein is an assay comprising at least one primer for detection of SARS-CoV-2 and at least primer for detection of a host gene associated with SARS-CoV-2.

Also disclosed is a method of simultaneously detecting SARS-CoV-2 and at least one host gene, the method comprising using at least one primer pair from Table 1 to detect SARS-CoV-2, and at least one primer pair from Table 2 to detect at least one host gene.

Also disclosed is a method of simultaneously detecting SARS-CoV-2 and at least one other respiratory virus, the method comprising using at least one primer pair from Table 1 to detect SARS-CoV-2, and at least one primer pair from Table 2 to detect at least one respiratory virus.

Further disclosed is a nucleic acid primer comprising any one of the SARS-CoV-2 primers of Table 1 or the primers of Table 2. Also disclosed is any combination of these primers. Disclosed are nucleic acids with 90% or more identity to the primers of Table 1 or Table 2.

Also disclosed is a pair of nucleic acid primers comprising a forward and reverse primer set selected from the SARS-CoV-2 primers of Table 1 or the primers of Table 2. Also disclosed is any combination of these primer pairs. Disclosed are primer pairs with 90% or more identity to the primers of Table 1 or Table 2.

Disclosed is a kit comprising any combination of the primers disclosed in Table 1 or Table 2, or primers with 90% or more identity to the primers of Table 1 or Table 2.

Also disclosed are methods of treating a subject in need thereof, based on the results of the assays disclosed herein.

### IV. BRIEF DESCRIPTION OF THE DRAWINGS

The accompanying drawings, which are incorporated in and constitute a part of this specification, illustrate several embodiments and together with the description illustrate the disclosed compositions and methods.

FIG. 1 shows an overview of an assay for host-virus SARS-CoV-2 characterization.

FIG. 2 shows integrated host-virus RNA sequencing assay. Host viral immune transcript are detected in tandem with SARS-CoV-2+ in nasal swabs. Induction of host inflammasome and interferon transcript, normalized to GUSB expression levels, correlate with viral titer.

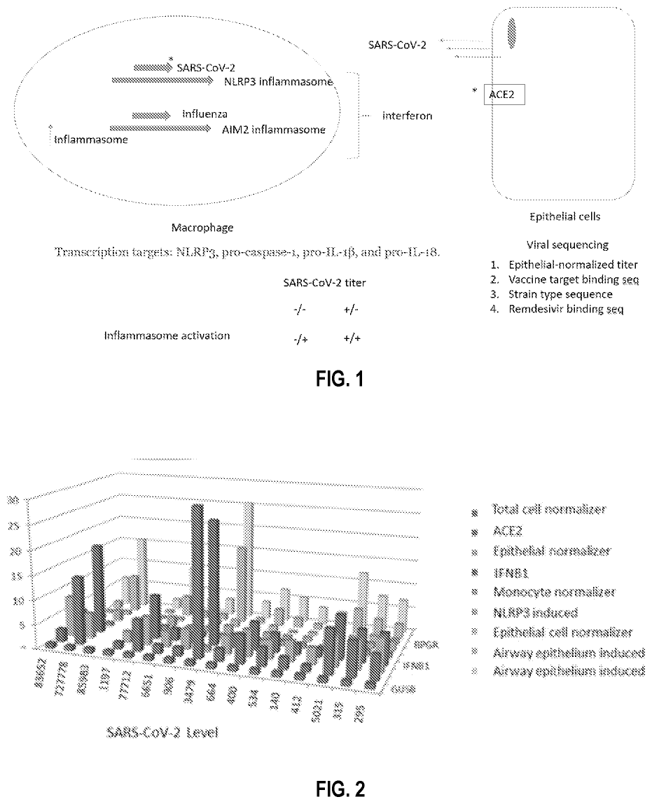

### V. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. Definitions

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

3′ end: The end of a nucleic acid molecule that does not have a nucleotide bound to it 3′ of the terminal residue.

5′ end: The end of a nucleic acid sequence where the 5′ position of the terminal residue is not bound by a nucleotide.

Amplification: A technique that increases the number of copies of a nucleic acid molecule (such as an RNA or DNA). An example of amplification is polymerase chain reaction (PCR), in which a sample is contacted with a pair of oligonucleotide primers under conditions that allow for the hybridization of the primers to a nucleic acid template in the sample. The primers are extended under suitable conditions (e.g., in the presence of a polymerase enzyme and dNTPs), dissociated from the template, re-annealed, extended, and dissociated to amplify the number of copies of the nucleic acid. The product of amplification can be characterized by electrophoresis, restriction endonuclease cleavage patterns, oligonucleotide hybridization or ligation, and/or nucleic acid sequencing using standard techniques.

Other examples of amplification include quantitative real-time polymerase chain reaction (qPCR), strand displacement amplification, as disclosed in U.S. Pat. No. 5,744,311; transcription-free isothermal amplification, as disclosed in U.S. Pat. No. 6,033,881; repair chain reaction amplification, as disclosed in PCT publication WO 90/01069; ligase chain reaction amplification, as disclosed in European patent publication EP-A-320,308; gap filling ligase chain reaction amplification, as disclosed in U.S. Pat. No. 5,427,930; and NASBA RNA transcription-free amplification, as disclosed in U.S. Pat. No. 6,025,134. Several embodiments include multiplex qPCR assays, which are useful for amplifying and detecting multiple nucleic acid sequences in a single reaction.

Biological sample: A sample of biological material obtained from a subject. Biological samples include all clinical samples useful for detection of disease or infection (e.g. a viral infection such as a SARS-CoV-2 infection) in subjects. Appropriate samples include any conventional biological samples, including clinical samples obtained from a human or veterinary subject. Exemplary samples include, without limitation, cells, cell lysates, blood smears, cytocentrifuge preparations, cytology smears, bodily fluids (e.g., blood, plasma, serum, saliva, sputum, urine, bronchial alveolar lavage, semen, cerebrospinal fluid (CSF), etc.), tissue biopsies or autopsies, fine-needle aspirates, and/or tissue sections. In a particular example, a biological sample is obtained from a nasal swab of a subject suspected of having SARS-CoV-2.

Complementary. Complementary binding occurs when the base of one nucleic acid molecule forms a hydrogen bond to the base of another nucleic acid molecule. Normally, the base adenine (A) is complementary to thymidine (T) and uracil (U), while cytosine (C) is complementary to guanine (G). For example, the sequence 5′-ATCG-3′ of one ssDNA molecule can bond to 3′-TAGC-5′ of another ssDNA to form a dsDNA. In this example, the sequence 5′-ATCG-3′ is the reverse complement of 3′-TAGC-5′.

Nucleic acid molecules can be complementary to each other even without complete hydrogen-bonding of all bases of each molecule. For example, hybridization with a complementary nucleic acid sequence can occur under conditions of differing stringency in which a complement will bind at some but not all nucleotide positions. In particular examples disclosed herein, the complementary sequence is complementary at a labeled nucleotide, and at each nucleotide immediately flanking the labeled nucleotide.

Consists of or consists essentially of: with regard to a polynucleotide (such as a probe or primer), a polynucleotide consists essentially of a specified nucleotide sequence if it does not include any additional nucleotides. However, the polynucleotide can include additional non-nucleic acid components, such as labels (for example, fluorescent, radioactive, or solid particle labels), sugars or lipids. With regard to a polynucleotide, a polynucleotide that consists of a specified nucleotide sequence does not include any additional nucleotides, nor does it include additional non-nucleic acid components, such as lipids, sugars or labels.

Contacting: Placement in direct physical association, for example solid, liquid or gaseous forms. Contacting includes, for example, direct physical association of fully- and partially-solvated molecules.

Control: A sample or standard used for comparison with an experimental sample. In some embodiments, the control is a sample obtained from a healthy patient, or a sample from a subject with SARS-CoV-2. In some embodiments, the control is a sample including SARS-CoV-2 nucleic acid. In other embodiments, the control is a biological sample obtained from a patient diagnosed with SARS-CoV-2. In still other embodiments, the control is a historical control or standard reference value or range of values (such as a previously tested control sample, such as a group of SARS-CoV-2 patients with known prognosis or outcome, or group of samples that represent baseline or normal values, such as the presence or absence of SARS-CoV-2 in a biological sample.

Ct (threshold cycle): The PCR cycle number at which the fluorescence emission (dRn) exceeds a chosen threshold, which is typically 10 times the standard deviation of the baseline (this threshold level can, however, be changed if desired). The threshold cycle is when the system begins to detect the increase in the signal associated with an exponential growth of PCR product during the log-linear phase. This phase provides information about the reaction. The slope of the log-linear phase is a reflection of the amplification efficiency. The efficiency of the reaction can be calculated by the following equation: E=10^{(1/slope)}, for example. The efficiency of the PCR should be 90-100% meaning doubling of the amplicon at each cycle. This corresponds to a slope of −3.1 to −3.6 in the Ct vs. log-template amount standard curve.

Detecting: To identify the existence, presence, or fact of something. General methods of detecting are known to the skilled artisan and may be supplemented with the protocols and reagents disclosed herein. For example, included herein are methods of detecting a virus (such as, for example, SARS-CoV-2) in a biological sample, including detecting a particular SARS-CoV-2 variant in a biological sample.

Diagnosis: The process of identifying a disease by its signs, symptoms and results of various tests. The conclusion reached through that process is also called “a diagnosis.” Forms of testing commonly performed include blood tests, medical imaging, urinalysis, and biopsy.

Isolated: An “isolated” biological component (such as a nucleic acid molecule or protein) has been substantially separated or purified away from other biological components in the cell of the organism in which the component naturally occurs. The term “isolated” does not require absolute purity. Nucleic acids and proteins that have been “isolated” include nucleic acids and proteins purified by standard purification methods. The term also embraces nucleic acids and proteins prepared by recombinant expression in a host cell, as well as chemically synthesized nucleic acids.

Mismatch nucleotide: a nucleotide that is not complementary to the corresponding nucleotide of the opposite polynucleotide strand.

Multiplex gPCR Amplification and detection of multiple nucleic acid species in a single qPCR reaction. By multiplexing, multiple target nucleic acids can be amplified in single tube. In some examples, multiplex PCR permits the simultaneous detection of multiple markers for SARS-CoV-2, and/or host genes, and/or other respiratory illnesses or naturally occurring microbes.

Nucleic acid: A deoxyribonucleotide or ribonucleotide polymer, which can include analogues of natural nucleotides that hybridize to nucleic acid molecules in a manner similar to naturally occurring nucleotides. In a particular example, a nucleic acid molecule is a single stranded (ss) DNA or RNA molecule, such as a probe or primer. In another particular example, a nucleic acid molecule is a double stranded (ds) nucleic acid, such as a target nucleic acid. Examples of modified nucleic acids are those with altered sugar moieties, such as a locked nucleic acid (LNA).

Nucleotide: The fundamental unit of nucleic acid molecules. A nucleotide includes a nitrogen-containing base attached to a pentose monosaccharide with one, two, or three phosphate groups attached by ester linkages to the saccharide moiety. The major nucleotides of DNA are deoxyadenosine 5′-triphosphate (dATP or A), deoxyguanosine 5′-triphosphate (dGTP or G), deoxycytidine 5′-triphosphate (dCTP or C) and deoxythymidine 5′-triphosphate (dTTP or T). The major nucleotides of RNA are adenosine 5′-triphosphate (ATP or A), guanosine 5′-triphosphate (GTP or G), cytidine 5′-triphosphate (CTP or C) and uridine 5′-triphosphate (UTP or U).

Conventional notation is used herein to describe nucleotide sequences: the left-hand end of a single-stranded nucleotide sequence is the 5′-end; the left-hand direction of a double-stranded nucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand;” sequences on the DNA strand having the same sequence as an mRNA transcribed from that DNA and which are located 5′ to the 5′-end of the RNA transcript are referred to as “upstream sequences;” sequences on the DNA strand having the same sequence as the RNA and which are 3′ to the 3′ end of the coding RNA transcript are referred to as “downstream sequences.” Unless denoted otherwise, whenever a polynucleotide sequence is represented, it will be understood that the nucleotides are in 5′ to 3′ orientation from left to right.

Oligonucleotide primers are nucleic acid molecules, usually DNA oligonucleotides of about 10-50 nucleotides in length (longer lengths are also possible). “Primers” are capable of supporting some type of enzymatic manipulation and which can hybridize with a target nucleic acid such that the enzymatic manipulation can occur. A primer can be made from any combination of nucleotides or nucleotide derivatives or analogs available in the art which do not interfere with the enzymatic manipulation. Typically, primers are at least about 10 nucleotides in length, such as at least about 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or about 50 nucleotides in length. For example, a primer can be about 10-50 nucleotides in length, such as, 12-15, 12-20, 12-25, 12-30, 12-35, 12-40, 12-45, 12-50, 14-15, 14-16, 14-18, 14-20, 14-25, 14-30, 15-16, 15-18, 15-20, 15-25, 15-30, 15-35, 15-40, 15-45, 15-50, 16-17, 16-18, 16-20, 16-22, 16-25, 16-30, 16-40, 16-50, 17-18, 17-20, 17-22, 17-25, 17-30, 18-19, 18-20, 18-22, 18-25, 18-30, 19-20, 19-22, 19-25, 19-30, 20-21, 20-22, 20-25, 20-30, 20-35, 20-40, 20-45, 20-50, 21-22, 21-25, 21-30, 22-25, 22-30, 22-40, 22-50, 23-24, 23-25, 23-30, 24-25, 24-30, 25-30, 25-35, 25-40 or 25-45, 25-50 nucleotides in length.

“Probes” are molecules capable of interacting with a target nucleic acid, typically in a sequence specific manner, for example through hybridization. The hybridization of nucleic acids is well understood in the art and discussed herein. Typically, a probe can be made from any combination of nucleotides or nucleotide derivatives or analogs available in the art.

Primers may be annealed to a complementary target DNA strand by nucleic acid hybridization to form a hybrid between the primer and the target DNA strand, and then extended along the target DNA strand by a DNA polymerase enzyme. One of skill in the art will appreciate that the hybridization specificity of a particular probe or primer typically increases with its length. Thus, for example, a probe or primer including 20 consecutive nucleotides typically will anneal to a target with a higher specificity than a corresponding probe or primer of only 15 nucleotides. In some embodiments, probes and primers are used in combination in a qPCR reaction.

Primer pair: Two primers (one “forward” and one “reverse”) that can be used for amplification of a nucleic acid sequence, for example by polymerase chain reaction (PCR) or other in vitro nucleic-acid amplification methods. The forward and reverse primers of a primer pair do not hybridize to overlapping complementary sequences on the target nucleic acid sequence.

Real-Time PCR (qPCR): A method for detecting and measuring products generated during each cycle of a PCR, which are proportionate to the amount of template nucleic acid prior to the start of PCR. The information obtained, such as an amplification curve, can be used to determine the presence of a target nucleic acid (such as a SARS-CoV-2 nucleic acid) and/or quantitate the initial amounts of a target nucleic acid sequence. Exemplary procedures for qPCR can be found in “Quantitation of DNA/RNA Using Real-Time PCR Detection” published by Perkin Elmer Applied Biosystems (1999); PCR Protocols (Academic Press, New York, 1989); A-Z of Quantitative PCR, Bustin (ed.), International University Line, La Jolla, Calif., 2004; and Quantitative Real-Time PCR in Applied Microbiology, Filion (Ed), Caister Academic Press, 2012.

Sensitivity and specificity: Statistical measurements of the performance of a binary classification test. Sensitivity measures the proportion of actual positives which are correctly identified (e.g., the percentage of samples that are identified as including nucleic acid from a particular virus). Specificity measures the proportion of negatives which are correctly identified (e.g., the percentage of samples that are identified as not including nucleic acid from a particular virus).

Sequence identity: The similarity between two nucleic acid sequences is expressed in terms of the similarity between the sequences, otherwise referred to as sequence identity.

Sequence identity is frequently measured in terms of percentage identity, similarity, or homology; a higher percentage identity indicates a higher degree of sequence similarity. The NCBI Basic Local Alignment Search Tool (BLAST), Altschul et al, J. Mol. Biol. 215:403-10, 1990, is available from several sources, including the National Center for Biotechnology Information (NCBI, Bethesda, Md.), for use in connection with the sequence analysis programs blastp, blastn, blastx, tblastn and tblastx. It can be accessed through the NCBI website. A description of how to determine sequence identity using this program is also available on the website.

When less than the entire sequence is being compared for sequence identity, homologs will typically possess at least 75% sequence identity over short windows of 10-20 amino acids, and can possess sequence identities of at least 85% or at least 90% or 95% depending on their similarity to the reference sequence. Methods for determining sequence identity over such short windows are described on the NCBI website.

These sequence identity ranges are provided for guidance only; it is entirely possible that strongly significant homologs could be obtained that fall outside of the ranges provided.

An alternative indication that two nucleic acid molecules are closely related is that the two molecules hybridize to each other under stringent conditions. Stringent conditions are sequence-dependent and are different under different environmental parameters. Generally, stringent conditions are selected to be about 5° C. to 20° C. lower than the thermal melting point (Tm) for the specific sequence at a defined ionic strength and pH. The Tm is the temperature (under defined ionic strength and pH) at which 50% of the target sequence hybridizes to a perfectly matched probe. Conditions for nucleic acid hybridization and calculation of stringencies can be found in Sambrook et al.; and Tijssen, Hybridization With Nucleic Acid Probes, Part I: Theory and Nucleic Acid Preparation, Laboratory Techniques in Biochemistry and Molecular Biology, Elsevier Science Ltd., 1993.

Signal: A detectable change or impulse in a physical property that provides information. In the context of the disclosed methods, examples include electromagnetic signals such as light, for example light of a particular quantity or wavelength. In certain examples, the signal is the disappearance of a physical event, such as quenching of light.

Subject: Any mammal, such as humans, non-human primates, pigs, sheep, horses, dogs, cats, cows, rodents and the like. In two non-limiting examples, a subject is a human subject or a murine subject. Thus, the term “subject” includes both human and veterinary subjects.

Target nucleic acid molecule: A nucleic acid molecule whose detection, quantitation, qualitative detection, or a combination thereof, is intended. The nucleic acid molecule need not be in a purified form. Various other nucleic acid molecules can also be present with the target nucleic acid molecule. For example, the target nucleic acid molecule can be a specific nucleic acid molecule (which can include RNA or DNA), the amplification of which is intended. In some examples, a target nucleic acid includes a region of the SARS-CoV-2 genome. Purification or isolation of the target nucleic acid molecule, if needed, can be conducted by methods known to those in the art, such as by using a commercially available purification kit or the like.

Under conditions sufficient for: A phrase that is used to describe any environment that permits a desired activity. In one example the desired activity is amplification of a nucleic acid molecule.

Wild-type: The genotype or phenotype that is most prevalent in nature. The naturally occurring, non-mutated version of a nucleic acid sequence. Among multiple alleles, the allele with the greatest frequency within the population is usually the wild-type. The term “native” can be used as a synonym for “wild-type.”

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. General

Disclosed herein is a molecular diagnostic assay and methods of detecting COVID-19 and related genes and conditions (termed “DXVX” for extended virus diagnostic assay) that utilizes rapid RNA sequencing by next-generation sequencing (semi-conductor sequencing method) to detect the virus but also provide data on a number of interacting host and viral factors as provide more rigorous quality control than existing SARS-CoV-2 assays.

The DXVX diagnostic assay is a single sequencing assay with multiple targets that provides deep coverage for improved sensitivity and specificity compared to PCR/TMA platforms. Assessment of adequacy of sampling can be done by including a set of targets for host genes that are highly expressed in the respiratory epithelium. This system can provide an estimate of viral load in SARS-CoV-2-positive samples by normalizing viral RNA levels to host targets that mark respiratory cells and measures total cellularity. It can also provide viral strain typing by assessing ten different polymorphous regions of the SARS-CoV-2 virus that distinguish sub-strains. It can assess for shifts in the sequence of both the human SARS-CoV-2 receptor (ACE2) and viral sequences in the spike (S) complex that mediate viral entry that may predict for changes in transmissibility and viral neutralizing immune responses, for example. It can also assess for mutations/variation in the Remdesivir binding site in the RdRp target that may influence drug activity. It can assess for coinfection with other common respiratory viruses, using targets for influenza A, influenza B, adenovirus, respiratory syncytial virus (RSV) and common non-SARS-like coronaviruses, or any other infectious agent or normally occurring microbes which may be present in the subject.

The DXVX assay can also detect the degree of immune activation by profiling host transcripts that are induced by coronaviruses infection within the epithelium and admixed inflammatory cells. Normalized transcript levels for the interferon and a set of genes associated with the NLRP3 and AIM2 inflammasome targets are used to provide correlates symptomology and virus titers (FIG. 2). These include MX1 and AIM2, produced by admixed respiratory epithelium and macrophages, and less-specific innate immune system activation, such as IFNB levels.

Although qPCR and TMA assays are discussed herein, this comprehensive multi-use SARS-CoV-2 diagnostic assay is superior for a variety of patient types. For example, this includes patients with low or equivocal or suspected false-negative qPCR/TMA results. The assessment of multiple viral targets, with definitive sequencing, overcomes the limitations of TMA and qPCR assays due to mutations in primer sets (false-negative) and low/equivocal positivity (false-positive due to contamination or probe breakdown). The inclusion of multiple sets of normalizers for total host/human cell content and specifically respiratory epithelium cell content allows flagging of SARS-CoV-2 negative test results that may represent inadequate sampling requiring recollection. The ability to derive host-normalized cell titers can provide an estimate of the magnitude of virus shedding at the sampled oronasal site for SARS-CoV-2-positive test results. Therefore, the DXVX assay can provide both accurate diagnosis and an estimate of viral levels in low/equivocal samples.

The system disclosed herein is also useful for patients with prolonged infection/failure to clear SARS-CoV-2 by qPCR assays. This allows for the assessment of whether levels of shedded virus are changing in response to therapy. It also allows a practitioner to assess shifts in viral sequence variants (particularly in the SARS-CoV-2 N, S1 and S2 genes) that may be contributing to persistent infection or assess for possible reinfection with a distinct substrain.

The system disclosed herein can replace full genome SARS-CoV-2 sequencing assays. Many laboratories have validated full SARS-CoV-2 sequencing assay by NGS methods. These provide full sequencing but are difficult to report rapidly due to need for additional bioinformatics time (or repeat sequencing) to resolve ambiguous or gapped sequences. Partial targeted SARS-CoV-2 sequencing for the core regions of the virus, as in DXVX, can efficiently type the virus strain without the need to resolve indeterminate sequence in non-essential regions resulting in 2-day turn-around time from extraction to result.

For COVID-19 patients undergoing new and targeted therapies, since the DXVX assay detects multiple key viral and host receptor sequences, functional prediction and correlations can be made about the virus population present in any given patient. This may be most important for patients treated with immune-mediated therapies such as convalescent plasma or post-vaccination where anti-Spike or anti-nucleocapsid viral neutralizing antibodies are key. Selection or outcome correlation with viral-targeted therapies, such as anti-protease agents, can be done based of the sequences at viral protease cleavage sites.

For patients with coinfection by multiple respiratory viruses, coinfection with SARS-CoV-2 and other viruses is generally detected by broad (and often non-specific) qPCR/TMA molecular respiratory panels. The DXVX assay can provide more complete sequencing information on the specific subtypes and levels of each of these co-occurring viruses and their persistence in follow-up sampling.

For COVID-19 patients considering immunomodulatory therapies, the DXVX assay can provide transcriptional correlates of the degree of viral-associated immune activation at the time of disease presentation at an accessible mucosal site. It can also monitor for effectiveness or non-response to anti-inflammatory therapies on the level of inflammasome activation.

The primers disclosed herein can have variation in the sequence thereof. For example, as applied to primers, a variant can have entire nucleotide sequence identity with the original primer, or alternatively, can have less than 100% nucleotide sequence identity with the primer. For example, a variant of a primer can have at least 90%, 95%, 98%, 99% or more identical in nucleotide sequence compare to the original primer. Phrased another way, the variant primer can vary by 1, 2, 3, 4, or 5 or more nucleotides compared to the original primer. Variant primers also include the entire original primer, and further comprising additional fused nucleotide sequences. Primer variants also includes nucleotides that are portions or subsequences of the parent primer, for example, unique subsequences (e.g., as determined by standard sequence comparison and alignment techniques) of the primers disclosed herein are also encompassed by the invention. One of skill will appreciate that many variants of the disclosed nucleotide sequences are encompassed by the invention.

The present disclosure provides compositions, kits, and methods for amplifying and detecting nucleic acid of SARS-CoV-2, as well as other infectious agents or host-associated microbes (such as viruses or bacteria), or alternatively/additionally amplifying nucleic acids encoding host cell expression proteins from a sample. Preferably, the samples are biological samples obtained from a human subject. The compositions, kits, and methods provide oligonucleotide sequences that recognize target sequences within the SARS-CoV-2 genome, including target sequences within the spike protein gene, for example, or the complement thereof. Other oligonucleotides may be used as probes for detecting amplified sequences of SARS-CoV-2 or other amplified products, or for capture of SARS-CoV-2 target nucleic acid or other amplified products.

The methods and compositions disclosed herein provide for the sensitive and specific detection of SARS-CoV-2 nucleic acids, as well as other infectious agents (such as viruses or bacteria), or alternatively/additionally amplifying host cell expression proteins from a sample. The methods include performing nucleic acid amplification of an SARS-CoV-2 target region and detecting the amplified product by, for example, specifically hybridizing the amplified product with a nucleic acid detection probe that provides a signal to indicate the presence of SARS-CoV-2 in the sample. The amplification step includes contacting the sample with one or more amplification oligomers specific for a target sequence in an SARS-CoV-2 target nucleic acid to produce an amplified product if SARS-CoV-2 nucleic acid is present in the sample. Amplification synthesizes additional copies of the target sequence or its complement by using at least one nucleic acid polymerase and an amplification oligomer to produce the copies from a template strand (e.g., by extending the sequence from a primer using the template strand).

Preferred compositions of the instant disclosure are configured to specifically hybridize to nucleic acid of SARS-CoV-2 or other target nucleic acids, with minimal cross-reactivity to other, non-target nucleic acids suspected of being in a sample (e.g., other viral pathogens or host proteins which are not targets). In some aspects, the compositions of the instant disclosure are configured to specifically hybridize to SARS-CoV-2 nucleic acid with minimal cross-reactivity to one or more non-SARS-CoV-2 pathogens (with the exception of those that are also targets). In one aspect, the compositions of the instant disclosure are part of a multiplex system that further includes components and methods of detecting one of more non-SARS-CoV-2 pathogens. For example, there can be compositions that independently amplify and detect the nucleic acids of SARS-CoV-2 and a host inflammasome in the same reaction. Amplified SARS-CoV-2 nucleic acids as well as other amplified nucleic acids can be detected using hybridization probes labeled with distinguishable fluorescent labels. In this way, SARS-CoV-2 can be detected in the multiplex amplification reaction independent of other target nucleic acids that also may be amplified in the multiplex reaction.

In certain aspects of the disclosure, a composition comprising at least two amplification oligomers is provided for determining the presence or absence of SARS-CoV-2 in a sample. Typically, the composition includes at least two amplification oligomers for amplifying a target region of a SARS-CoV-2 target nucleic acid, wherein said oligomers correspond to the sequences found in Table 1—SEQ ID NOS: 1-30. In such embodiments, at least one amplification oligomer comprises a forward primer, and the other is a reverse primer. Also disclosed are primers in Table 2, represented by SEQ ID NOS: 31-80. These primers can amplify other targets. kir

In certain embodiments, the composition is provided as an aqueous or dried formulation for amplification of SARS-CoV-2 nucleic acid, or a reaction mixture comprising or reconstituted from such a formulation.

The present invention can utilize real-time reverse transcriptase polymerase chain reaction (rRT-PCR) assay to detect the presence of SARS-CoV-2 in clinical samples. rRT-PCR assays are well known and widely deployed in diagnostic virology (see, e.g., Pang, J. et al. (2020) “Potential Rapid Diagnostics, Vaccine and Therapeutics for 2019 Novel Coronavirus (2019-nCoV): A Systematic Review,” J. Clin. Med. 26; 9(3)E623 doi: 10.3390/jcm9030623; Kralik, P. et al. (2017) “A Basic Guide to Real-Time PCR in Microbial Diagnostics: Definitions, Parameters, and Everything,” Front. Microbiol. 8:108. doi: 10.3389/fmicb.2017.00108).

To more easily describe the rRT-PCR assays of the present invention, such assays may be envisioned as involving multiple reaction steps: (1) the reverse transcription of SARS-CoV-2 RNA that may be present in the clinical sample that is to be evaluated for SARS-CoV-2 presence; (2) the PCR-mediated amplification of the SARS-CoV-2 cDNA produced from such reverse transcription; (3) the hybridization of SARS-CoV-2-specific probes to such amplification products; (4) the double-strand-dependent 5″→3″ exonuclease cleavage of the hybridized SARS-CoV-2-specific probes; and (5) the detection of the unquenched probe fluorophores signifying that the evaluated clinical sample contained SARS-CoV-2.

It will be understood that such steps may be conducted separately (for example, in two or more reaction chambers, or with reagents for the different steps being added at differing times, etc.). However, it is preferred that such steps are to be conducted within the same reaction chamber, and that all reagents needed for the rRT-PCR assays of the present invention are to be provided to the reaction chamber at the start of the assay. It will also be understood that although the polymerase chain reaction (PCR) (see, e.g. Ghannam, M. G. et al. (2020) “Biochemistry, Polymerase Chain Reaction (PCR),” StatPearls Publishing, Treasure Is.; pp 0.1-4; Lorenz, T. C. (2012) “Polymerase Chain Reaction: Basic Protocol Plus Troubleshooting And Optimization Strategies,” J. Vis. Exp. 2012 May 22; (63):e3998; pp. 1-15) is the preferred method of amplifying SARS-CoV-2 cDNA produced via reverse transcription, other DNA amplification technologies could alternatively be employed.

Preferably, such Forward and Reverse primers will permit the amplification of a desired region of the target, such as the “S” protein of SARS-CoV-2, which encodes the virus spike surface glycoprotein and is required for host cell targeting. The SARS-CoV-2 spike surface glycoprotein is a key protein for specifically characterizing a coronavirus as being SARS-CoV-2 (Chen, Y. et al. (2020) “Structure Analysis Of The Receptor Binding Of 2019-Ncov,” Biochem. Biophys. Res. Commun. 525:135-140; Masters, P. S. (2006) “The Molecular Biology Of Coronaviruses,” Adv. Virus Res. 66:193-292). The amplification of any one SARS-CoV-2 target alone is sufficient for the specific determination of SARS-CoV-2 presence in clinical samples.

The presence of such amplified molecules is preferably detected in real time during amplification. While qPCR is the preferred method, such detection can be accomplished using any suitable method, e.g., molecular beacon probes, scorpion primer-probes, TaqMan probes, etc. (Navarro, E. et al. (2015) “Real-Time PCR Detection Chemistry,” Clin. Chim Acta 439:231-250). All of these methods employ an oligonucleotide that is labeled with a fluorophore and complexed to a quencher of the fluorescence of that fluorophore.

In several embodiments, the oligonucleotide probe can be labeled, for example with a base-linked or terminally-linked fluorophore and non-fluorescent quencher for use in qPCR assays. Fluorophores for use in qPCR assays are known in the art. They can be obtained, for example, from Life Technologies (Gaithersburg, Md.), Sigma-Genosys (The Woodlands, Tex.), Genset Corp. (La Jolla, Calif.), or Synthetic Genetics (San Diego, Calif.). Fluorophores can be conjugated to the oligonucleotides, for example by post-synthesis modification of oligonucleotides that are synthesized with reactive groups linked to bases. Useful fluorophores include: fluorescein, fluorescein isothiocyanate (FITC), carboxy tetrachloro fluorescein (TET), NHS-fluorescein, 5 and/or 6-carboxy fluorescein (FAM), 5-(or 6-) iodoacetamidofluorescein, 5-{[2(and 3)-5-(Acetylmercapto)-succinyl]amino}fluorescein (SAMSA-fluorescein), 5′-hexachloro-fluorescein (HEX), 6-carboxy-4′,5′-dichloro-2′,7′ dimethoxyfluorescein,succinimidyl ester (JOE) and other fluorescein derivatives, rhodamine, Lissamine rhodamine B sulfonyl chloride, Texas red sulfonyl chloride, 5 and/or 6 carboxy rhodamine (ROX) and other rhodamine derivatives, coumarin, 7-amino-methyl-coumarin, 7-Amino-4-methylcoumarin-3-acetic acid (AMCA), and other coumarin derivatives, BODIPY fluorophores, Cascade Blue fluorophores such as 8-methoxypyrene-1,3,6-trisulfonic acid trisodium salt, Lucifer yellow fluorophores such as 3,6-Disulfonate-4-amino-naphthalimide, phycobiliproteins derivatives, Alexa fluor dyes (available from Molecular Probes, Eugene, Oreg.) and other fluorophores known to those of skill in the art. For a general listing of useful fluorophores, see also Hermanson, G. T., BIOCONJUGATE TECHNIQUES (Academic Press, San Diego, 1996).

Quenchers for use in qPCR assays are also known in the art and include, for example, 6-carboxytetramethylrhodamine,succinidyl ester (6-TAMRA; TAMRA) and “non-fluorescent quencher (NFP)” for use with TAQMAN™ probes available from Life technologies.

Several embodiments include the use of PCR and/or qPCR. PCR reaction conditions typically include either two or three step cycles. Two step cycles have a denaturation step followed by a hybridization/elongation step. Three step cycles include a denaturation step followed by a hybridization step during which the primer hybridizes to the strands of DNA, followed by a separate elongation step. The polymerase reactions are incubated under conditions in which the primers hybridize to the target sequences and are extended by a polymerase. The amplification reaction cycle conditions are selected so that the primers hybridize specifically to the target sequence and are extended.

Primers are typically designed so that all of the primers participating in a particular reaction have melting temperatures that are within at least five degrees Celsius, and more typically within two degrees Celsius of each other. Primers are further designed to avoid priming on themselves or each other. Primer concentration should be sufficient to bind to the amount of target sequences that are amplified so as to provide an accurate assessment of the quantity of amplified sequence. Those of skill in the art will recognize that the amount of concentration of primer will vary according to the binding affinity of the primers as well as the quantity of sequence to be bound. Typical primer concentrations will range from 0.01 μM to 0.5 μM.

In a typical PCR cycle, a sample including a DNA polynucleotide and a PCR reaction cocktail is denatured by treatment in thermal cycler at about 90-98° C. for 10-90 seconds. The denatured polynucleotide is then hybridized to oligonucleotide primers by treatment in a thermal cycler at a temperature of about 30-65° C. for 1-2 minutes. Chain extension then occurs by the action of a DNA polymerase on the polynucleotide annealed to the oligonucleotide primer. This reaction occurs at a temperature of about 70-75° C. for 30 seconds to 5 minutes. Any desired number of PCR cycles may be carried out depending on variables including but not limited to the amount of the initial DNA polynucleotide, the length of the desired product and primer stringency. The above temperature ranges and the other numbers are exemplary and not intended to be limiting. These ranges are dependent on other factors such as the type of enzyme, the type of container or plate, the type of biological sample, the size of samples, etc. One of ordinary skill in the art will recognize that the temperatures, time durations and cycle number can readily be modified as necessary.

Several embodiments include quantitative real-time polymerase chain reaction (qPCR), which is used to simultaneously quantify and amplify a specific part of a given nucleic acid molecule. It is used, for example, to determine whether or not a specific sequence is present in the sample; and if it is present, the number of copies in the sample.

qPCR monitors the fluorescence emitted during the reaction as an indicator of amplicon production during each PCR cycle, as opposed to endpoint detection. The real-time progress of the reaction can be viewed in some systems. Typically, real-time PCR uses the detection of a fluorescent reporter. Typically, the fluorescent reporter's signal increases in direct proportion to the amount of PCR product in a reaction. By recording the amount of fluorescence emission at each cycle, it is possible to monitor the PCR reaction during exponential phase where the first significant increase in the amount of PCR product correlates to the initial amount of target template. The higher the starting copy number of the nucleic acid target, the sooner a significant increase in fluorescence is observed. Thus, the procedure follows the general pattern of polymerase chain reaction, but the nucleic acid molecule is quantified after each round of amplification. In several embodiments the amplified nucleic acid molecule is quantified by the use of fluorescent dye that intercalates with double-strand DNA. In other embodiments (e.g., when multiplex qPCR assays are utilized) amplified nucleic acid molecule is quantified by use of oligonucleotide probes labeled with a reporter fluorophore that can be detected in the qPCR assay.

In certain embodiments, the amplified products are directly visualized with detectable label such as a fluorescent DNA-binding dye. In one embodiment the amplified products are quantified using an intercalating dye, including but not limited to SYBR green, SYBR blue, DAPI, propidium iodine, Hoeste, SYBR gold, ethidium bromide, acridines, proflavine, acridine orange, acriflavine, fluorcoumanin, ellipticine, daunomycin, chloroquine, distamycin D, chromomycin, homidium, mithramycin, ruthenium polypyridyls, anthramycin. For example, a DNA binding dye such as SYBR green binds double stranded DNA and an increase in fluorescence intensity can be measured. For example, the fluorescent dsDNA dye can be added to the buffer used for a PCR reaction. The PCR assay can be performed in a thermal cycler, and after each cycle, the levels of fluorescence are measured with a detector, such as a camera. The dye fluoresces much more strongly when bound to dsDNA (e.g., amplified PCR product). Because the amount of the dye intercalated into the double-stranded DNA molecules is typically proportional to the amount of the amplified DNA products, the amount of amplified nucleic acid can be quantified by detecting the fluorescence of the intercalated dye using detection instruments known in the art. When referenced to a standard dilution, the dsDNA concentration in the PCR can be determined.

In addition to various kinds of fluorescent DNA-binding dye, other luminescent labels such as sequence specific oligonucleotide probes can be employed in the amplification reaction to facilitate the detection and quantification of the amplified product. Probe based quantitative amplification relies on the sequence-specific detection of a desired amplified product. Unlike the dye-based quantitative methods, it utilizes target-specific probe labeled with a detectable marker such as a base-linked or terminally-linked fluorophore and quencher. Such markers are known to the person of ordinary skill in the art and described herein. Further, methods for performing probe-based quantitative amplification are well established in the art (see, e.g., U.S. Pat. No. 5,210,015).

For detection using oligonucleotide probes, the reaction is prepared as usual for PCR conditions, with the addition of the sequence specific labeled oligonucleotide probe. After denaturation of the DNA, the labeled probe is able to bind to its complementary sequence in the region of interest of the template DNA. When the PCR reaction is heated to the proper extension temperature, the polymerase is activated and DNA extension proceeds. As the polymerization continues it reaches the labeled probe bound to the complementary sequence of DNA. The polymerase breaks the probe into separate nucleotides, and separates the fluorescent reporter from the quencher. This results in an increase in fluorescence as detected by the optical assembly. As PCR cycle number increases more and more of the fluorescent reporter is liberated from its quencher, resulting in a well-defined geometric increase in fluorescence. This allows accurate determination of the final, and initial, quantities of DNA.

In one embodiment, the fluorescently-labeled probes (such as probes disclosed herein) rely upon fluorescence resonance energy transfer (FRET), or in a change in the fluorescence emission wavelength of a sample, as a method to detect hybridization of a DNA probe to the amplified target nucleic acid in real-time. For example, FRET that occurs between fluorogenic labels on different probes (for example, using HybProbes) or between a donor fluorophore and an acceptor or quencher fluorophore on the same probe (for example, using a molecular beacon or a TAQMAN™ probe) can identify a probe that specifically hybridizes to the DNA sequence of interest. In some embodiments, the fluorescently-labeled DNA probes used to identify amplification products have spectrally distinct emission wavelengths, thus allowing them to be distinguished within the same reaction tube, for example in multiplex PCR, such as a multiplex real-time PCR.

Any type of thermal cycler apparatus can be used for the amplification of target nucleic acids as described above and/or the determination of hybridization. Examples of suitable apparatuses include PTC-100® Peltier Thermal Cycler (MJ Research, Inc.; San Francisco, Calif.), a ROBOCYCLER® 40 Temperature Cycler (Agilent/Stratagene; Santa Clara, Calif.), or GeneAmp® PCR System 9700 (Applied Biosystems; Foster City, Calif.). For real-time PCR, any type of real-time thermocycler apparatus can be used. For example, ICYCLER IQ™ or CFX96™ real-time detection systems (Bio-Rad, Hercules, Calif.), LIGHTCYCLER® systems (Roche, Mannheim, Germany), a 7700 Sequence Detector (Perkin Elmer/Applied Biosystems; Foster City, Calif.), ABI™ systems such as the 7000, 7300, 7500, 7700, or 7900 systems (Applied Biosystems; Foster City, Calif.), or an MX4000™, MX3000™ or MX3005™ qPCR system (Agilent/Stratagene; Santa Clara, Calif.), DNA ENGINE OPTICON® Continuous Fluorescence Detection System (Bio-Rad, Hercules, Calif.), ROTOR-GENE® Q real-time cycler (Qiagen, Valencia, Calif.), or SMARTCYCLER® system (Cepheid, Sunnyvale, Calif.) can be used to amplify and detect nucleic acid sequences in real-time. In some embodiments, real-time PCR is performed using a TAQMAN® array format, for example, a microfluidic card in which each well is pre-loaded with primers and probes for a particular target. The reaction is initiated by adding a sample including nucleic acids and assay reagents (such as a PCR master mix) and running the reactions in a real-time thermocycler apparatus.

In several embodiments, multiplex qPCR assays are used to amplify and detect the first and second nucleic acid molecules as described above. In the multiplex qPCR assays, the first and second nucleic acids are amplified and detected in a single reaction. For example, the multiplex qPCR assay can include amplifying from a biological sample SARS-CoV-2 using primers found in Table 1. The qPCR can also include amplifying from a biological sample a host gene, or another virus, as found in Tables 2 and 4, respectively.

In some embodiments, multiplex qPCR is performed using the Applied Biosystem 7500 Real-time PCR system and (ABI) Gene Expression Master mix (Cat #4369016). In one example, the multiplex real-time PCR can be performed in a total reaction volume of 50 μl containing 10 μl of DNA extract, 40 μl of 2×PCR master mix, the forward and reverse primers for the first oligonucleotide primer pair, the forward and reverse primers of the second oligonucleotide primer pair, and the first and second oligonucleotide probes corresponding to the first and second nucleic acid molecules amplified by the first and second oligonucleotide primer pairs, respectively. The concentration of both sets of primers can be 300 nM and the probes can be 200 nM. In some examples, the following protocol can be used for the multiplex qPCR: 50° C. for 2 minutes; 95° C. for 10 minutes; followed by 40 cycles of 95° C. for 15 seconds and 60° C. for 1 minute. Each multiplex qPCR assay can include a standard dilution series for DNA quantification. Further, samples can be analyzed in duplicate as part of a CLIA protocol.

In some embodiments, amplification and detection of the first and second nucleic acid sequences in the multiplex qPCR assays can be performed using any of the oligonucleotide pairs and probes provided herein for amplification and detection of the first and second nucleic acid molecules, such as those described above and in Tables 1-4 below.

Detection of the first and second nucleic acid sequences in multiplex qPCR assays is performed using a first oligonucleotide probes specific for the first nucleic acid sequence and a second oligonucleotide probe specific for the second nucleic acid sequence. For example, detection of the first nucleic acid molecule can include use of an oligonucleotide probe including a nucleotide sequence set forth as nucleotides 139-158 of SEQ ID NO: 1, or the complement thereof, and detection of the second nucleic acid molecule can include use of an oligonucleotide probe including a nucleotide sequence set forth as nucleotides 4315-4330 of SEQ ID NO: 1, or the complement thereof.

In multiplex qPCR assays, the oligonucleotide probes used for detecting the first and second nucleic acid molecules are labeled with detectable markers that can be differentially detected in the same assay using detection equipment available to the person of ordinary skill in the art. For example, the oligonucleotide probe for detecting the first nucleic acid can be labeled with a first fluorophore and quencher and the oligonucleotide probe for detecting the second nucleic acid can be labeled with a second fluorophore and quencher, wherein the first and second base-linked or terminally-linked fluorophore and quencher can be differentially detected.

In one example, the first and second oligonucleotide probes are labeled with TAQMAN™ fluorophores and quenchers that can be differentially detected, such as fluorophores and quenchers available from Applied Biosystems by Life Technologies, Carlsbad, Calif.). In one example, the oligonucleotide probe for detecting the first nucleic acid sequence is labeled with the VIC fluorophore and the NFQ™ quencher available from Applied Biosystems by Life Technologies, Carlsbad, Calif., and the oligonucleotide probe for detecting the second nucleic acid is labeled with the 6-carboxyfluorescin (FAM) fluorophore and the NFQ™ quencher available from Applied Biosystems by Life Technologies, Carlsbad, Calif.

Also provided are kits for practicing the methods as described herein. A kit in accordance with the present disclosure include a packaged combination of one or both of (1) an oligomer combination as described herein for amplification of a SARS-CoV-2 target nucleic acid, and (2) one or more oligomer combinations as described herein for amplification of an additional target nucleic acid. In some embodiments, any oligomer combination described herein is present in the kit. The kits may further include a number of optional components such as, for example, capture probes (e.g., poly-(k) capture probes as described in US 2013/0209992). Other reagents that may be present in the kits include reagents suitable for performing in vitro amplification such as buffers, salt solutions, appropriate nucleotide triphosphates (e.g., dATP, dCTP, dGTP, dTTP; and/or ATP, CTP, GTP and UTP), and/or enzymes (e.g., a thermostable DNA polymerase, or a reverse transcriptase and/or RNA polymerase). Oligomers as described herein can be packaged in a variety of different embodiments, and those skilled in the art will appreciate that the disclosure embraces many different kit configurations. For example, a kit optionally can include amplification oligomers for only one target region of a SARS-CoV-2 genome, as well as amplification oligomers for a host factor, or it may include amplification oligomers for multiple SARS-CoV-2 target regions, or multiple other targets. In addition, for a kit that includes a detection probe together with an amplification oligomer combination, selection of amplification oligomers and detection probe oligomers for a kit are linked by a common target region, the kit will include a probe that binds to a sequence amplifiable by an amplification oligomer combination of the kit. In certain embodiments, the kit further includes a set of instructions for practicing methods in accordance with the present disclosure, where the instructions may be associated with a package insert and/or the packaging of the kit or the components thereof.

Also disclosed are methods of using the assays to detect disease, as well as methods of treating a subject based on the results of said assays.

### C. Example: Methods for SARS-CoV-2 Assay

Extracted RNA from nasal or oropharyngeal swabs was used at the substrate. Custom primer design was performed for all sequencing targeted using phylogeny analysis (for viral sequencing) and cross-dataset analysis for host target (see Tables 1-3). Multiplex PCR, PCR product bar coding, purification, amplification and quantification (library prep), and library normalization was performed on the Ion Chef/S5 or Genexus instrument platforms (ThermoFisher). Emulsion PCR took place on the Ion Chef or Genexus and NGS sequencing on the Ion Torrent S5 or emulsion PCR and sequencing took place on the Genexus platforms (ThermoFisher/LifeTech) Base-calling, alignment, variant calling and initial annotation occurred of the VCF using Ion Tools and SARS-CoV-2 FASTA and alignment plugins. Reanalysis of BAM and FASTA files were done using custom-developed software tools. Using standard multivariate statistical tools (ANOVA, ANCOVA regression) and clustering algorithms, development of mucosal immune response predictor was performed using in-house training data sets. The model correlates the level of transcriptional activation for the genes in Table 3 with levels of serum cytokine levels and humoral response in parallel samples. Validation of assay performance was done using data sets in Table 3, with cutoff for immune prediction score established by second training set using receiver-operator curve analysis with the immune predictor score and serum cytokine levels as readout.

### Phylogeny Analysis Method:

SARS-CoV-2 phylogeny methods using partial genome sequencing: custom analytic methods were developed to match the existing strain typing (e.g. NextStrain/GISAID) with a custom NGS analytic pipeline using the limited viral targets included in DXVX 1.0. These include combining programs multiple sequence alignment (MAFFT), with maximal likelihood tree building functions with oversample bootstrapping analysis.

TABLE 1
Custom CoV-2 Primer Sets in included in DXVX 1.0 Panel.

| | CUSTOM | | CUSTOM | | |
|---|---|---|---|---|---|
| SEQUENCING | FORWARD | SEQUENCE | REVERSE | SEQUENCE | |
| LOCATION | PRIMER | ID NO: | PRIMER | ID NO: | INDICATION |
| 241 VAR | AACCAACTTTCG | 1 | CGTTGAAACCAG | 2 | Strain typing |
| | ATCTCTTGUAG | | GGACAAGGCUC | | |
| 1059 VAR | GAAATTGCTTGG | 3 | TTTGGTTGCATT | 4 | Strain typing |
| | UACACGGAACG | | CATTTGGUGAC | | |
| 3037 VAR | GGCATTGATTTA | 5 | TCTAACCAATCT | 6 | Strain typing |
| | GATGAGUGGAG | | TCTTCTTGCUC | | |
| 8782 VAR | TATTGATGGTGG | 7 | GATGTTGCAAAG | 8 | Strain typing |
| | TGTCACTCGUG | | TCAGTGTACUC | | |
| 25563 VAR | CAATTGGAACTG | 9 | AGCAAGTTGCAA | 10 | Strain typing |
| | TAACTTUGAAG | | ACAAAGUGAAC | | |
| 26144 VAR | GAACATGTTACC | 11 | ACTAGCAAGAAU | 12 | Strain typing |
| | TTCTTCATCUA | | ACCACGAAAGC | | |
| 27964 VAR | CTTGTCACGCCT | 13 | ATGGGTGATTTA | 14 | Strain typing |
| | AAACGAACAUG | | GAACCAGCCUC | | |
| 28883 VAR | AACTTCCTCAAG | 15 | GATTTCTTAGTG | 16 | Strain typing |
| | GAACAACAUTG | | ACAGTTUGGCC | | |
| RdRp target | AGCCTTACATTA | 17 | CCTAGCTCTCTG | 18 | RdRp assay |
| | AGTGGGATUTG | | AAGTGGTAUCC | | comparisons |
| RdRp-RBS | TATTACTAGATA | 19 | AAGTACTTATCA | 20 | Remdesivir |
| | AACGCACUACG | | ACAACTUCAAC | | binding |
| Spike 614 | CCAACAATTTGG | 21 | GCCAAGTAGGAG | 22 | Strain typing |
| | CAGAGACAUTG | | TAAGTTGAUC | | |
| Spike RBD/ | CACTTCTGGTTG | 23 | ATTGCACCAAAA | 24 | Transmissibi- |
| fusion | GACCTTTGGUG | | TTGGAGCUAAG | | lity |
| | | | | | mutation |
| Spike S1-S2 | TGTTTATTCTAC | 25 | CTGAATTTTCTG | 26 | Pathogeni- |
| junction | AGGTTCTAAUG | | CACCAAGUGAC | | city |
| | | | | | mutations |
| N1 (CDC | CATCGATATCGG | 27 | GCGCCCCACTGC | 28 | qPCR assay |
| region) | TAATTAUACAG | | GTTCTCCAUTC | | comparison |
| N2 (CDC | GCTGCTGAGGCT | 29 | CCTGTGTAGGTC | 30 | qPCR assay |
| region) | TCUAAGAAG | | AACCACGTUCC | | comparison |

### VI. TABLES

TABLE 2
Additional elements of the DXVX 1.0 sequencing panel.

| | |
|---|---|
| SEQUENCE | SEQUENCE |
| ID | ID |
| NO: | NO: |

Inflammasome Sensor Score

| | | | | | |
|---|---|---|---|---|---|
| AIM2 | CTTGCACTCTAAGGA | 31 | GAGGTTGTAGTCAG | 32 | Inflammasome |
| | AGCUGAG | | TGGAGATUG | | score |
| IFNB1 | GCAATTGAATGGGAG | 33 | GCCAGGAGGTTCTC | 34 | Immune |
| (DNA/ | GCTUG | | AACAAUA | | readout |
| RNA) | | | | | |
| MX1 | GCCACAGCAACTCCA | 35 | CCTCCCAGAGGAGT | 36 | Inflammasome |
| | TCTUA | | AGGATUAT | | score |
| RPP25 | AGCAAGCCTCTTGGA | 37 | CTCACAAACCGGCAG | 38 | Inflammasome |
| | AAUCT | | TCUAA | | score |
| GUSB | CCATTCCTATGCCATC | 39 | GTATTGGATGGTCCC | 40 | Normalizer |
| | GUGT | | TGGUG | | |

Host Receptor Interacting Domain and Epithelium Measures
for Viral Level/Sample Quality Control

| | | | | | |
|---|---|---|---|---|---|
| ACE2 SNP | ACCCAAGTTCAAAGG | 41 | GGACATTCTCTTCAG | 42 | RNA/DNA |
| 26 | CTGAUAAG | | TAATATUG | | receptor type |
| ACE2 SNP | GATGTCCCGGAGCCG | 43 | TCTTCCGATCTCTGA | 44 | RNA/DNA |
| 720 | TATCAAUG | | TCCCAGUG | | receptor type |
| ACE2 | TTTCTCTACAGGGAG | 45 | TGGTTAGGAGGTCC | 46 | Receptor RNA |
| E17-18 | GAGGAUG | | AAGUGT | | normalization |
| RPGR E4-5 | AATTAGCTGCCTGUG | 47 | CAGTTAGGGCAGCT | 48 | Respiratory |
| | GAAGG | | GAAGTAUT | | epithelium QC |
| | | | | | & viral |
| | | | | | pseudotiter |
| FOXJ1 | CTACAAGTGGAUCAC | 49 | AGTCGCCGCTTCTUG | 50 | Respiratory |
| E2-3 | GGACAA | | AAA | | epithelium QC |
| | | | | | & viral |
| | | | | | pseudotiter |
| C14orf | AACTGCTGACAATGC | 51 | GGTAATCATCATGAC | 52 | Respiratory |
| E5-6 | AACUAAA | | GCTGAAUC | | epithelium QC |
| | | | | | & viral |
| | | | | | pseudotiter |
| C14orf E8 | GCTGCTCAAATTCTGC | 53 | ATGCCAGAAGTGTAT | 54 | RNA-DNA |
| (RNA/ | GATUG | | GGTUCC | | normalizer |
| DNA)_ | | | | | pseudotiter |
| | | | | | calculation |
| LRP1 | CCTGCAGAGATCAAA | 55 | CCTCATCTGAGCCGU | 56 | Host |
| | TAACCTGTAUC | | CCA | | expression |
| | | | | | normalizer/ |
| | | | | | QC |

Respiratory Virus Panel

| | | | | | |
|---|---|---|---|---|---|
| HAdV_ | GATGGCTACCCCTTCG | 57 | TCAGGCTGAAGTAC | 58 | Adenovirus |
| pan | AUGA | | GTCUCG | | |
| (custom) | | | | | |
| Hadv | GCCCCAGTGGTCTTAC | 59 | GCCACGGTGGGGTT | 60 | CDC |
| pan (CDC) | ATGCACAUC | | TCTAAACUT | | comparison |
| HRSV_ | TTAACCAGCAAAGTG | 61 | TGATCATTTGTTATA | 62 | Respiratory |
| pan | TUAGA | | GGCATATCATUG | | syncytial virus |
| (custom) | | | | | (RSV) |
| HRSV | AACAGTTTAACATTAC | 63 | TCATTGACTTGAGAT | 64 | CDC |
| pan (CDC) | CAAGUGA | | ATTGAUGC | | comparison |
| HCOV-229E | TCGGAATCCTTCAAG | 65 | GGCTTTTGCATTTCA | 66 | Non-SARS |
| (custom) | UGACA | | TGCUT | | Coronavirus |
| HCoV- | CTCGACCACACTGACT | 67 | ACGGTTGTTACUGAC | 68 | Non-SARS |
| HKU1 | GGUG | | GCAAG | | Coronavirus |
| (custom) | | | | | |
| HCoV-NL63 | GGTGTTCCTGACAATT | 69 | CAGAAGCATTTAAGC | 70 | Non-SARS |
| (custom) | CTUCAAC | | CTATTUCA | | Coronavirus |
| HCoV- | TGATATGCATTGGGG | 71 | CCATTTCCTGGATTG | 72 | Non-SARS |
| OC43 | TGAUG | | GUCAG | | Coronavirus |
| (custom) | | | | | |
| 65 | CTGAGTGATGCCCCA | 73 | GGGCATTAGCAUGA | 74 | Type A |
| Influenza | TUCCT | | ACCAGT | | influenza |
| A NS1 | | | | | |
| (custom) | | | | | |
| Influenza | CTAAGGGCTTUCACC | 75 | CCCATTCTCATTACT | 76 | CDC |
| A_NS1 | GAAGA | | GCTUC | | comparison |
| (CDC) | | | | | |
| Influenza_ | GACCAGAGTGGAAG | 77 | TTCTGGTGATAATCG | 78 | Type B |
| B_NS1 | GCTUGT | | GTGCUC | | influenza |
| (custom) | | | | | |
| Influenza | ATGGCCATCGGATCC | 79 | TGTCAGCTATTATGG | 80 | CDC |
| B NS1 | UCAAC | | AGCUG | | comparison |
| (CDC) | | | | | |

TABLE 3
Elements of the DXVX 1.0 Assay Validation.

| Assay element | Referent/Comparison Method(s) | | Samples |
|---|---|---|---|
| Accuracy of CoV-2 | 1. | Comparison with viral reads obtained from the | 80+, 20− |
| infection | | existing JML full SARS-CoV-2 clinical assay | |
| | 2. | Comparison with positive/negative/equivocal | |
| | | results from the clinical PCR assay for SARS- | |
| | | CoV-2 assay (ABI 7500 RT-PCR). | |
| Accuracy of CoV-2 | 1. | Dilution studies using recombinant SARS- | 2 sets of virus |
| virus levels | | CoV-2 viral control sequences diluted in | controls |
| (“pseudo-titer’) | | respiratory epithelium cell line background | |
| Detection of | 1. | Comparison of viruses detected in the blinded | 20 coinfected |
| respiratory virus | | respiratory specimens using the BioFire | and 20 non- |
| microbiota | | FilmArray Respiratory Panel with DXVX on a | coinfected SARS- |
| | | parallel samples obtained for SARS-CoV-2 PCR | CoV-2+ |
| | 2. | Comparison of custom-developed pan-virus | |
| | | developed primer sets with CDC consensus | |
| | | primer sequences for influenza A/B, adenovirus | |
| | | and RSV | |
| Inflammasome | 1. | Comparison of DXVX activation score with | 40 high cytokine |
| activation | | serum cytokine levels (TNF, IL6 and soluble | and 40 low- |
| | | IL2R) measure on parallel samples from | cytokine samples |

TABLE 4
Validation of other respiratory virus by the DXVX
assay using complete genomic reference standards.

| | | | | DXCX | Target | DXVX reads |
|---|---|---|---|---|---|---|
| | Material | Expected level | Detected | reads per | length | (bases/read |
| Virus | tested | count/ul | by DXVX | region | (bp) | length) |
| Corona_OC43 | Full | 25.0 | YES | 2991 | 123 | 24.3 |
| | genome | | | | | |
| Corona_229E | Full | 500.0 | YES | 1220 | 201 | 6.1 |
| | genome | | | | | |
| FLU_A_H1N1 | Full | 100.0 | YES | 3385 | 158 | 21.4 |
| | genome | | | | | |
| FLU_B | Full | 500.0 | YES | 2024 | 90 | 22.5 |
| | genome | | | | | |
| RSV_B | Full | 10.0 | YES | 158 | 157 | 1 |
| | genome | | | | | |

1. An assay comprising at least one primer for detection of SARS-CoV-2 and at least primer for detection of a host gene associated with SARS-CoV-2.

2. The assay of claim 1, wherein the host gene encodes an inflammasome signature in mucosal samples is indicative of systemic immune response to coronavirus.

3. The assay of claim 1, wherein at least one set of primers is used to detect SARS-CoV-2.

4. The assay of claim 3, wherein at least two sets of primers are used to detect SARS-CoV-2.

5. The assay of claim 1, wherein the at least two sets of primers for detecting SARS-CoV-2 are selected from the primers of Table 1.

6. The assay of claim 1, wherein the assay further comprises at least one primer for detecting other respiratory viruses; wherein the respiratory virus is not SARS-CoV-2.

7. The assay of claim 6, wherein the at least one primer targets respiratory viruses that consist of influenza A, influenza B, adenovirus, respiratory syncytial virus (RSV) and common non-SARS-like coronaviruses.

8. The assay of claim 6, wherein the at least one primer is selected from the primers of the respiratory virus panel of Table 2.

9. The assay of claim 1, wherein the host gene is an epithelial marker.

10. The assay of claim 9, wherein the epithelial marker is selected from Table 2.

11. The assay of claim 2, wherein the inflammasome is selected from the inflammasomes of Table 2.

12. A method of simultaneously detecting SARS-CoV-2 and at least one host gene, the method comprising using at least one primer pair from Table 1 to detect SARS-CoV-2, and at least one primer pair from Table 2 to detect at least one host gene.

13. A nucleic acid primer comprising any one of the SARS-CoV-2 primers of Table 1.

14. (canceled)

15. (canceled)

16. A nucleic acid primer of claim 13, comprising at least 90% identity to any one of the SARS-CoV-2 primers of Table 1.

17. (canceled)

18. (canceled)

19. A nucleic acid primer comprising any one of the primers of Table 2.

20. (canceled)

21. (canceled)

22. A nucleic acid primer of claim 19, comprising at least 90% identity to any one of the primers of Table 2.

23. (canceled)

24. (canceled)

25. (canceled)

26. (canceled)


SEQUENCE LISTING (80 sequences)

SEQ ID NO: 1 - DNA, 23 bp, Artificial Sequence
  Primer
aaccaactttcgatctcttguag

SEQ ID NO: 2 - DNA, 23 bp, Artificial Sequence
  primer
cgttgaaaccagggacaaggcuc

SEQ ID NO: 3 - DNA, 23 bp, Artificial Sequence
  primer
gaaattgcttgguacacggaacg

SEQ ID NO: 4 - DNA, 23 bp, Artificial Sequence
  primer
tttggttgcattcatttggugac

SEQ ID NO: 5 - DNA, 23 bp, Artificial Sequence
  primer
ggcattgatttagatgaguggag

SEQ ID NO: 6 - DNA, 23 bp, Artificial Sequence
  primer
tctaaccaatcttcttcttgcuc

SEQ ID NO: 7 - DNA, 23 bp, Artificial Sequence
  primer
tattgatggtggtgtcactcgug

SEQ ID NO: 8 - DNA, 23 bp, Artificial Sequence
  primer
gatgttgcaaagtcagtgtacuc

SEQ ID NO: 9 - DNA, 23 bp, Artificial Sequence
  primer
caattggaactgtaacttugaag

SEQ ID NO: 10 - DNA, 23 bp, Artificial Sequence
  primer
agcaagttgcaaacaaagugaac

SEQ ID NO: 11 - DNA, 23 bp, Artificial Sequence
  primer
gaacatgttaccttcttcatcua

SEQ ID NO: 12 - DNA, 23 bp, Artificial Sequence
  primer
actagcaagaauaccacgaaagc

SEQ ID NO: 13 - DNA, 23 bp, Artificial Sequence
  primer
cttgtcacgcctaaacgaacaug

SEQ ID NO: 14 - DNA, 23 bp, Artificial Sequence
  primer
atgggtgatttagaaccagccuc

SEQ ID NO: 15 - DNA, 23 bp, Artificial Sequence
  primer
aacttcctcaaggaacaacautg

SEQ ID NO: 16 - DNA, 23 bp, Artificial Sequence
  primer
gatttcttagtgacagttuggcc

SEQ ID NO: 17 - DNA, 23 bp, Artificial Sequence
  primer
agccttacattaagtgggatutg

SEQ ID NO: 18 - DNA, 23 bp, Artificial Sequence
  primer
cctagctctctgaagtggtaucc

SEQ ID NO: 19 - DNA, 23 bp, Artificial Sequence
  primer
tattactagataaacgcacuacg

SEQ ID NO: 20 - DNA, 23 bp, Artificial Sequence
  primer
aagtacttatcaacaactucaac

SEQ ID NO: 21 - DNA, 23 bp, Artificial Sequence
  primer
ccaacaatttggcagagacautg

SEQ ID NO: 22 - DNA, 22 bp, Artificial Sequence
  primer
gccaagtaggagtaagttgauc

SEQ ID NO: 23 - DNA, 23 bp, Artificial Sequence
  primer
cacttctggttggacctttggug

SEQ ID NO: 24 - DNA, 23 bp, Artificial Sequence
  primer
attgcaccaaaattggagcuaag

SEQ ID NO: 25 - DNA, 23 bp, Artificial Sequence
  primer
tgtttattctacaggttctaaug

SEQ ID NO: 26 - DNA, 23 bp, Artificial Sequence
  primer
ctgaattttctgcaccaagugac

SEQ ID NO: 27 - DNA, 23 bp, Artificial Sequence
  primer
catcgatatcggtaattauacag

SEQ ID NO: 28 - DNA, 23 bp, Artificial Sequence
  primer
gcgccccactgcgttctccautc

SEQ ID NO: 29 - DNA, 21 bp, Artificial Sequence
  primer
gctgctgaggcttcuaagaag

SEQ ID NO: 30 - DNA, 23 bp, Artificial Sequence
  primer
cctgtgtaggtcaaccacgtucc

SEQ ID NO: 31 - DNA, 22 bp, Artificial Sequence
  primer
cttgcactctaaggaagcugag

SEQ ID NO: 32 - DNA, 23 bp, Artificial Sequence
  primer
gaggttgtagtcagtggagatug

SEQ ID NO: 33 - DNA, 20 bp, Artificial Sequence
  primer
gcaattgaatgggaggctug

SEQ ID NO: 34 - DNA, 21 bp, Artificial Sequence
  primer
gccaggaggttctcaacaaua

SEQ ID NO: 35 - DNA, 20 bp, Artificial Sequence
  primer
gccacagcaactccatctua

SEQ ID NO: 36 - DNA, 22 bp, Artificial Sequence
  primer
cctcccagaggagtaggatuat

SEQ ID NO: 37 - DNA, 20 bp, Artificial Sequence
  primer
agcaagcctcttggaaauct

SEQ ID NO: 38 - DNA, 20 bp, Artificial Sequence
  primer
ctcacaaaccggcagtcuaa

SEQ ID NO: 39 - DNA, 20 bp, Artificial Sequence
  primer
ccattcctatgccatcgugt

SEQ ID NO: 40 - DNA, 20 bp, Artificial Sequence
  primer
gtattggatggtccctggug

SEQ ID NO: 41 - DNA, 23 bp, Artificial Sequence
  primer
acccaagttcaaaggctgauaag

SEQ ID NO: 42 - DNA, 23 bp, Artificial Sequence
  primer
ggacattctcttcagtaatatug

SEQ ID NO: 43 - DNA, 23 bp, Artificial Sequence
  primer
gatgtcccggagccgtatcaaug

SEQ ID NO: 44 - DNA, 23 bp, Artificial Sequence
  primer
tcttccgatctctgatcccagug

SEQ ID NO: 45 - DNA, 22 bp, Artificial Sequence
  primer
tttctctacagggaggaggaug

SEQ ID NO: 46 - DNA, 20 bp, Artificial Sequence
  primer
tggttaggaggtccaagugt

SEQ ID NO: 47 - DNA, 20 bp, Artificial Sequence
  primer
aattagctgcctguggaagg

SEQ ID NO: 48 - DNA, 22 bp, Artificial Sequence
  primer
cagttagggcagctgaagtaut

SEQ ID NO: 49 - DNA, 21 bp, Artificial Sequence
  primer
ctacaagtggaucacggacaa

SEQ ID NO: 50 - DNA, 18 bp, Artificial Sequence
  primer
agtcgccgcttctugaaa

SEQ ID NO: 51 - DNA, 22 bp, Artificial Sequence
  primer
aactgctgacaatgcaacuaaa

SEQ ID NO: 52 - DNA, 23 bp, Artificial Sequence
  primer
ggtaatcatcatgacgctgaauc

SEQ ID NO: 53 - DNA, 21 bp, Artificial Sequence
  primer
gctgctcaaattctgcgatug

SEQ ID NO: 54 - DNA, 21 bp, Artificial Sequence
  primer
atgccagaagtgtatggtucc

SEQ ID NO: 55 - DNA, 26 bp, Artificial Sequence
  primer
cctgcagagatcaaataacctgtauc

SEQ ID NO: 56 - DNA, 18 bp, Artificial Sequence
  primer
cctcatctgagccgucca

SEQ ID NO: 57 - DNA, 20 bp, Artificial Sequence
  primer
gatggctaccccttcgauga

SEQ ID NO: 58 - DNA, 20 bp, Artificial Sequence
  primer
tcaggctgaagtacgtcucg

SEQ ID NO: 59 - DNA, 25 bp, Artificial Sequence
  primer
gccccagtggtcttacatgcacauc

SEQ ID NO: 60 - DNA, 23 bp, Artificial Sequence
  primer
gccacggtggggtttctaaacut

SEQ ID NO: 61 - DNA, 20 bp, Artificial Sequence
  primer
ttaaccagcaaagtgtuaga

SEQ ID NO: 62 - DNA, 27 bp, Artificial Sequence
  primer
tgatcatttgttataggcatatcatug

SEQ ID NO: 63 - DNA, 23 bp, Artificial Sequence
  primer
aacagtttaacattaccaaguga

SEQ ID NO: 64 - DNA, 23 bp, Artificial Sequence
  primer
tcattgacttgagatattgaugc

SEQ ID NO: 65 - DNA, 20 bp, Artificial Sequence
  primer
tcggaatccttcaagugaca

SEQ ID NO: 66 - DNA, 20 bp, Artificial Sequence
  primer
ggcttttgcatttcatgcut

SEQ ID NO: 67 - DNA, 20 bp, Artificial Sequence
  primer
ctcgaccacactgactggug

SEQ ID NO: 68 - DNA, 20 bp, Artificial Sequence
  primer
acggttgttacugacgcaag

SEQ ID NO: 69 - DNA, 23 bp, Artificial Sequence
  primer
ggtgttcctgacaattctucaac

SEQ ID NO: 70 - DNA, 23 bp, Artificial Sequence
  primer
cagaagcatttaagcctattuca

SEQ ID NO: 71 - DNA, 20 bp, Artificial Sequence
  primer
tgatatgcattggggtgaug

SEQ ID NO: 72 - DNA, 20 bp, Artificial Sequence
  primer
ccatttcctggattggucag

SEQ ID NO: 73 - DNA, 20 bp, Artificial Sequence
  primer
ctgagtgatgccccatucct

SEQ ID NO: 74 - DNA, 20 bp, Artificial Sequence
  primer
gggcattagcaugaaccagt

SEQ ID NO: 75 - DNA, 20 bp, Artificial Sequence
  primer
ctaagggcttucaccgaaga

SEQ ID NO: 76 - DNA, 20 bp, Artificial Sequence
  primer
cccattctcattactgctuc

SEQ ID NO: 77 - DNA, 20 bp, Artificial Sequence
  primer
gaccagagtggaaggctugt

SEQ ID NO: 78 - DNA, 21 bp, Artificial Sequence
  primer
ttctggtgataatcggtgcuc

SEQ ID NO: 79 - DNA, 20 bp, Artificial Sequence
  primer
atggccatcggatccucaac

SEQ ID NO: 80 - DNA, 20 bp, Artificial Sequence
  primer
tgtcagctattatggagcug